(19) United States
(12) Patent Application Publication
(10) Pub. No.: US 2017/0071874 A1
(43) Pub. Date: Mar. 16, 2017

(54) TOPICAL ANALGESIC

(71) Applicant: Tania Aranki, Lenexa, KS (US)

(72) Inventor: Tania Aranki, Lenexa, KS (US)

(21) Appl. No.: 15/260,984

(22) Filed: Sep. 9, 2016

Related U.S. Application Data
(60) Provisional application No. 62/216,840, filed on Sep. 10, 2015.

(51) Int. Cl. A61K 31/045; A61K 36/534; A61K 9/00; A61K 36/61; A61K 9/06; A61K 36/752; A61K 47/44; A61K 36/53

(52) U.S. Cl. CPC A61K 31/045; A61K 47/44; A61K 36/534; A61K 36/53; A61K 36/61; A61K 9/06; A61K 36/752; A61K 9/0014

(57) ABSTRACT

One or more of embodiments of the present invention generally concern a topical analgesic, which can be used to treat painful skin ailments. The topical analgesic can comprise 8 to 40 weight percent of methanol and 0.05 to 5 weight percent of one or more essential oils selected from the group consisting of spearmint essential oil, lavender essential oil, eucalyptus essential oil, lemon essential oil, peppermint essential oil, basil essential oil, and wintergreen essential oil. Furthermore, the topical analgesic can comprise less than 1 weight percent of parabens and less than 1 weight percent of gluten. In addition, the topical analgesic can meet one or more of the following criteria: (i) the topical analgesic comprises less than 10 weight percent of methyl salicylate and (2) the topical analgesic comprises less than 3 weight percent of camphor.

### RELATED APPLICATIONS

This application claims the priority benefit under 35 U.S.C. §119(e) of U.S. Provisional Patent Application Ser. No. 62/216,840 entitled “TOPICAL ANALGESIC,” filed Sep. 10, 2015, the entire disclosure of which is incorporated herein by reference.

### BACKGROUND

1. Field of the Invention

The present invention is generally related to analgesic compositions. More particularly, the present invention is generally related to topical analgesic compositions and methods for using such compositions in treating pain.

2. Description of the Related Art

Generally, analgesics are considered a group of drugs used to achieve analgesia or relief from pain. Exemplary analgesics found in today's market can include topical analgesics, which are applied to the skin of a person in order to relieve pain from one or more ailments. Topical analgesics are available in a wide variety of formulations. However, existing topical analgesics typically have one or more of the following drawbacks: (1) inadequate pain relief; (2) short duration of pain relief; (3) unpleasant odors; (4) harmful toxins and/or allergens; and/or (5) greasy and unpleasant consistencies.

Therefore, there is a need for topical analgesics that do not exhibit the above-listed deficiencies.

### SUMMARY

One or more of embodiments of the present invention generally concern a topical analgesic. The topical analgesic can comprise 8 to 40 weight percent of methanol and 0.05 to 5 weight percent of one or more essential oils selected from the group consisting of spearmint essential oil, lavender essential oil, eucalyptus essential oil, lemon essential oil, peppermint essential oil, basil essential oil, and wintergreen essential oil. Furthermore, the topical analgesic can comprise less than 1 weight percent of parabens and less than 1 weight percent of gluten. In addition, the topical analgesic can meet one or more of the following criteria: (i) the topical analgesic comprises less than 10 weight percent of methyl salicylate; and (2) the topical analgesic comprises less than 3 weight percent of camphor.

One or more of embodiments of the present invention generally concern a packaged topical analgesic comprising a topical analgesic and a container for housing the topical analgesic. The topical analgesic can comprise 8 to 40 weight percent of methanol and 0.05 to 5 weight percent of one or more essential oils selected from the group consisting of spearmint essential oil, lavender essential oil, eucalyptus essential oil, lemon essential oil, peppermint essential oil, basil essential oil, and wintergreen essential oil. Furthermore, the topical analgesic can comprise less than 1 weight percent of parabens and less than 1 weight percent of gluten. In addition, the topical analgesic can meet one or more of the following criteria: (i) the topical analgesic comprises less than 10 weight percent of methyl salicylate; and (2) the topical analgesic comprises less than 3 weight percent of camphor.

One or more of embodiments of the present invention generally concern a solid stick topical analgesic. Generally, the solid stick topical analgesic comprises: (i) 8 to 40 weight percent menthol; (ii) 0.05 to 5 weight percent of one or more essential oils selected from the group consisting of spearmint essential oil, lavender essential oil, eucalyptus essential oil, lemon essential oil, peppermint essential oil, basil essential oil, and wintergreen essential oil; (iii) 10 to 80 weight percent of kokum butter; (iv) 5 to 70 weight percent of a carrier oil; (v) 2 to 40 weight percent of a beeswax; (vi) 0.5 to 10 weight percent of Vitamin E; (vii) less than 10 weight percent of methyl salicylate; (viii) less than 1 weight percent of parabens; (ix) less than 1 weight percent of gluten; and (x) less than 3 percent of camphor.

### BRIEF DESCRIPTION OF THE FIGURES

Embodiments of the present invention are described herein with reference to the following drawing figures, wherein:

FIG. 1 depicts a container comprising the analgesic composition in stick form according to various embodiments of the present invention.

### DETAILED DESCRIPTION

The present invention generally relates to novel topical analgesic formulations that: (1) provide superior, long-lasting pain relief; (2) have a pleasant odor and consistency; (3) do not contain any harmful toxins or allergens; and (4) include wellness-promoting essential oils. As discussed below, the topical analgesics of the present invention can comprise various types of ingredients that provide these desirable properties. The various types of ingredients that may be used to produce the topical analgesics of the present invention are discussed in detail below.

In various embodiments, the topical analgesic can comprise methanol. For example, the topical analgesics can comprise 8 to 40, 10 to 25, or 12 to 14 weight percent of methanol.

In various embodiments, the topical analgesics can comprise one or more essential oils. In certain embodiments, the topical analgesics can comprise at least one, two, three, four, five, or six essential oils selected from the group consisting of spearmint essential oil, lavender essential oil, eucalyptus essential oil, lemon essential oil, peppermint essential oil, basil essential oil, and wintergreen essential oil. In one or more embodiments, the topical analgesics can comprise 0.05 to 5.0, 0.1 to 4.0, or 0.75 to 2.0 weight percent of one or more essential oils selected from the group consisting of spearmint essential oil, lavender essential oil, eucalyptus essential oil, lemon essential oil, peppermint essential oil, basil essential oil, and wintergreen essential oil.

In various embodiments, the topical analgesic can comprise less than 1, 0.5, or 0.1 weight percent of parabens. In certain embodiments, the topical analgesic can comprise no parabens.

In various embodiments, the topical analgesic can comprise less than 1, 0.5, or 0.1 weight percent of gluten. In certain embodiments, the topical analgesic can comprise no gluten.

In addition, in various embodiments, the topical analgesic can meet one or more of the following criteria: (i) the topical analgesic can comprise less than 10, 5, or 1 weight percent of methyl salicylate; and (2) the topical analgesic can comprise less than 3, 2, 1, or 0.5 weight percent of camphor. In certain embodiments, the topical analgesic can comprise no methyl salicylate and/or no camphor.

In various embodiments, the topical analgesic can comprise any one of the following ingredients in an amount of less than 1, 0.5, or 0.1 weight percent: artificial dyes, artificial fragrances, carbomers, petrolatum, sodium lauryl sulfate, sodium cetearyl sulfate, ethyl alcohol, stearyl alcohol, triethanolamine stearate, and/or stearyl alcohol. In certain embodiments, the topical analgesic can comprise no artificial dyes, no artificial fragrances, no carbomers, no petrolatum, no sodium lauryl sulfate, no sodium cetearyl sulfate, no ethyl alcohol, no stearyl alcohol, no triethanolamine stearate, and/or no stearyl alcohol.

Generally, the form of the topical analgesic will influence the type of ingredients used to form the topical analgesic. In various embodiments, the topical analgesic may be produced in the form of a cream, an ointment, a gel, or a solid stick.

The following tables (TABLES 1-6) list the ingredients of several types of topical analgesics formed in accordance with various embodiments (1A, 1B, 1C, 1D, 1E, 2A, 2B, 2C, 2D, 2E, 3A, 3B, 3C, 3D, 4A, 4B, 4C, 4D, 5A, 5B, 5B, 5D, 6A, 6B, 6C, and 6D) of the present invention. In the below tables, the amount of each ingredient is provided as a weight percentage of the total topical analgesic formulation. Further, in the below tables, “Opt” means that the ingredient is an optional component of that particular embodiment. Finally, when the amount of a particular ingredient is listed as being less than (“<”) a certain value, it means that particular ingredient can be entirely absent from the composition or it can be present in an amount up to the recited value.

TABLE 1, below, lists the possible ingredients for the topical analgesic, which are generic for all the possible forms of the topical analgesic (e.g., creams, ointments, gels, or solid sticks).

TABLE 1
Generic to All Forms of the Topical Analgesic

| | Topical Analgesic Formulation | | | | |
|---|---|---|---|---|---|
| INGREDIENTS | 1A | 1B | 1C | 1D | 1E |
| Menthol | 8-40 | 8-40 | 10-25 | 11-16 | 12-14 |
| Total Essential Oils (all types) | 0.05-5 | 0.05-5 | 0.1-4 | 0.5-3 | 0.75-2 |
| Eucalyptus Oil | Opt | Opt | Opt | Opt | Opt |
| Spearmint Oil | Opt | Opt | Opt | Opt | Opt |
| Lavender Oil | Opt | Opt | Opt | Opt | Opt |
| Lemon Oil | Opt | Opt | Opt | Opt | Opt |
| Peppermint Oil | Opt | Opt | Opt | Opt | Opt |
| Basil Oil | Opt | Opt | Opt | Opt | Opt |
| Wintergreen | Opt | Opt | Opt | Opt | Opt |
| Capsaicin | Opt | Opt | Opt | Opt | Opt |
| Kokum Butter | Opt | Opt | Opt | Opt | Opt |
| Carrier Oil (Avocado or Apricot Kernel) | Opt | Opt | Opt | Opt | Opt |
| Beeswax | Opt | Opt | Opt | Opt | Opt |
| Vitamin E (MT-50) | Opt | Opt | Opt | Opt | Opt |
| Witch Hazel (Alcohol Free) | Opt | Opt | Opt | Opt | Opt |
| Mineral Salts | Opt | Opt | Opt | Opt | Opt |
| Isopropyl Alcohol | Opt | Opt | Opt | Opt | Opt |
| Guar Gum | Opt | Opt | Opt | Opt | Opt |
| Water | Opt | Opt | Opt | Opt | Opt |
| Methyl Salicylate | <10 | Opt | <5 | <1 | 0.0 |
| Camphor | <3 | Opt | <2 | <0.5 | 0.0 |
| Parabens | <1 | Opt | <0.5 | <0.1 | 0.0 |
| Gluten | <1 | Opt | <0.5 | <0.1 | 0.0 |
| Total Artificial Colorants | Opt | <1 | <0.5 | <0.1 | 0.0 |
| Total Petroleum Derived Compounds | Opt | <1 | <0.5 | <0.1 | 0.0 |
| Artificial Fragrance | Opt | <1 | <0.5 | <0.1 | 0.0 |
| Carbomers | Opt | <1 | <0.5 | <0.1 | 0.0 |
| Total Alcohols | Opt | <1 | <0.5 | <0.1 | 0.0 |
| FD&C blue #1 | Opt | <1 | <0.5 | <0.1 | 0.0 |
| FD&C yellow #5 | Opt | <1 | <0.5 | <0.1 | 0.0 |
| Petrolatum | Opt | <1 | <0.5 | <0.1 | 0.0 |
| Stearyl Alcohol | Opt | <1 | <0.5 | <0.1 | 0.0 |
| Cetostearyl Alcohol | Opt | <1 | <0.5 | <0.1 | 0.0 |
| SD Alcohol 40 | Opt | <1 | <0.5 | <0.1 | 0.0 |
| Propylene Glycol | Opt | <1 | <0.5 | <0.1 | 0.0 |
| Arnica Montana Flower Extract (Arnica) | Opt | <1 | <0.5 | <0.1 | 0.0 |
| Aloe Barbadensis Leaf Extract (Aloe) | Opt | <1 | <0.5 | <0.1 | 0.0 |
| Boswellia Carterii Resin Extract (Boswellia) | Opt | <1 | <0.5 | <0.1 | 0.0 |
| Calendula Officinalis Flower Extract | Opt | <1 | <0.5 | <0.1 | 0.0 |
| (Calendula) | | | | | |
| Camellia Sinensis Leaf Extract (Green Tea) | Opt | <1 | <0.5 | <0.1 | 0.0 |
| Arctium Lappa Root Extract (Burdock) | Opt | <1 | <0.5 | <0.1 | 0.0 |
| Herbal Extract (ILEX Paraguariensis) | Opt | <1 | <0.5 | <0.1 | 0.0 |
| Carthamus Tinctorius Seed Oil (Safflower) | Opt | <1 | <0.5 | <0.1 | 0.0 |
| Chamomila Recutita Flower Oil (Matricaria) | Opt | <1 | <0.5 | <0.1 | 0.0 |
| Lavandula Angustifolia Oil (Lavender) | Opt | <1 | <0.5 | <0.1 | 0.0 |
| Rosmarinus Officinalis Leaf Oil (Rosemary) | Opt | <1 | <0.5 | <0.1 | 0.0 |
| Allantoin (Comfrey Root) | Opt | <1 | <0.5 | <0.1 | 0.0 |
| Aloe Vera (Aloe Barbadensis) Leaf Juice | Opt | <1 | <0.5 | <0.1 | 0.0 |
| Thyme (Thymus Vulgaris) Oil | Opt | <1 | <0.5 | <0.1 | 0.0 |
| Juniper Berry | Opt | <1 | <0.5 | <0.1 | 0.0 |
| White Tea | Opt | <1 | <0.5 | <0.1 | 0.0 |
| Cedar Leaf Oil | Opt | <1 | <0.5 | <0.1 | 0.0 |
| Nutmeg Oil | Opt | <1 | <0.5 | <0.1 | 0.0 |
| Sorbitan Stearate | Opt | <1 | <0.5 | <0.1 | 0.0 |
| Disteareth-75 IPDI | Opt | <1 | <0.5 | <0.1 | 0.0 |
| Glyceryl Stearate | Opt | <1 | <0.5 | <0.1 | 0.0 |
| Potassium Stearate | Opt | <1 | <0.5 | <0.1 | 0.0 |
| Triethanolamine Stearate | Opt | <1 | <0.5 | <0.1 | 0.0 |
| Stearic Acid | Opt | <1 | <0.5 | <0.1 | 0.0 |
| PET-150 Distearate | Opt | <1 | <0.5 | <0.1 | 0.0 |
| Steareth | Opt | <1 | <0.5 | <0.1 | 0.0 |
| PEG 40 Hydrogentated Caster Oil | Opt | <1 | <0.5 | <0.1 | 0.0 |
| Light Mineral Oil | Opt | <1 | <0.5 | <0.1 | 0.0 |
| Turpentine Oil | Opt | <1 | <0.5 | <0.1 | 0.0 |
| Polysorbate 60 | Opt | <1 | <0.5 | <0.1 | 0.0 |
| Polysorbate 80 | Opt | <1 | <0.5 | <0.1 | 0.0 |
| Glycerin | Opt | <1 | <0.5 | <0.1 | 0.0 |
| Isononanoate PEG-7 caprylic/capric | Opt | <1 | <0.5 | <0.1 | 0.0 |
| Glycerides | | | | | |
| Thymol | Opt | <1 | <0.5 | <0.1 | 0.0 |
| Diisopropyl Adipate | Opt | <1 | <0.5 | <0.1 | 0.0 |
| Aminomethyl Propanol | Opt | <1 | <0.5 | <0.1 | 0.0 |
| DMDM Hydantoin | Opt | <1 | <0.5 | <0.1 | 0.0 |
| sodium Hydroxide | Opt | <1 | <0.5 | <0.1 | 0.0 |
| Echinacea | Opt | <1 | <0.5 | <0.1 | 0.0 |
| Isopropyl Myristate | Opt | <1 | <0.5 | <0.1 | 0.0 |
| Acrylates/C10 30 Alkyl Acrylate | Opt | <1 | <0.5 | <0.1 | 0.0 |
| Crosspolymer | | | | | |
| Dimethyl Sulfone | Opt | <1 | <0.5 | <0.1 | 0.0 |
| Silicon Dioxide | Opt | <1 | <0.5 | <0.1 | 0.0 |
| Triethanolamine | Opt | <1 | <0.5 | <0.1 | 0.0 |
| Trolamine | Opt | <1 | <0.5 | <0.1 | 0.0 |
| Isoceteh 20 | Opt | <1 | <0.5 | <0.1 | 0.0 |
| Poloxamer 407 | Opt | <1 | <0.5 | <0.1 | 0.0 |
| Quaternium 15 | Opt | <1 | <0.5 | <0.1 | 0.0 |
| Sodium Lauryl Sulfate | Opt | <1 | <0.5 | <0.1 | 0.0 |
| Titanium Dioxide | Opt | <1 | <0.5 | <0.1 | 0.0 |
| Lanolin | Opt | <1 | <0.5 | <0.1 | 0.0 |
| Quaternium 15 | Opt | <1 | <0.5 | <0.1 | 0.0 |
| Sodium Cetearyl Sulfate | Opt | <1 | <0.5 | <0.1 | 0.0 |
| Edetate Disodium | Opt | <1 | <0.5 | <0.1 | 0.0 |
| Ceresin | Opt | <1 | <0.5 | <0.1 | 0.0 |
| Cyclomethicone | Opt | <1 | <0.5 | <0.1 | 0.0 |
| Paraffin | Opt | <1 | <0.5 | <0.1 | 0.0 |
| Microcrystalline Wax | Opt | <1 | <0.5 | <0.1 | 0.0 |

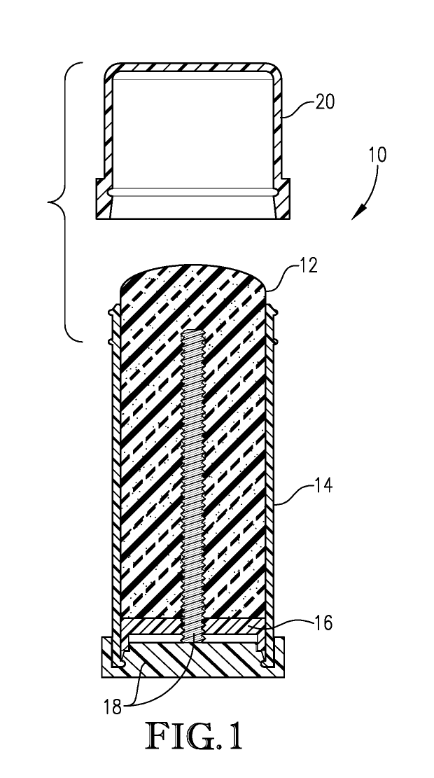

TABLE 2, below, lists the possible ingredients for the topical analgesic in the form of a solid stick. An exemplary solid stick configuration is demonstrated in FIG. 1. As shown in FIG. 1, a solid stick package 10 comprises a solid stick of the topical analgesic 12 housed in a container 14. The user can pick up the container 14 and apply the solid stick of topical analgesic 12 directly on the desired skin surface. The container 14 also contains a base 16 that the solid stick of topical analgesic may be positioned and a screw mechanism 18 to further push the topical analgesic 12 up the container via the base 16, thereby further exposing more of the solid stick. In addition, the solid stick package can also contain a cap 20 to seal the topical analgesic 12 within the container 14. The container 14 can be any container known or commonly used in the pharmaceutical arts. For example, the container can comprise a plastic or metal container configured to house a solid stick of the topical analgesic.

The ranges listed in TABLE 2 are generic for all solid sticks formed from the topical analgesic of the present invention.

TABLE 2
Generic to Stick Formulations

| | Topical Analgesic Formulations | | | | |
|---|---|---|---|---|---|
| INGREDIENTS | 2A | 2B | 2C | 2D | 2E |
| Menthol | 8-40 | 8-40 | 10-25 | 11-16 | 12-14 |
| Total Essential Oils (all types) | 0.05-5 | 0.05-5 | 0.1-4 | 0.5-3 | 0.75-2 |
| Eucalyptus Oil | Opt | Opt | Opt | Opt | Opt |
| Spearmint Oil | Opt | Opt | Opt | Opt | Opt |
| Lavender Oil | Opt | Opt | Opt | Opt | Opt |
| Lemon Oil | Opt | Opt | Opt | Opt | Opt |
| Peppermint Oil | Opt | Opt | Opt | Opt | Opt |
| Basil Oil | Opt | Opt | Opt | Opt | Opt |
| Wintergreen | Opt | Opt | Opt | Opt | Opt |
| Capsaicin | Opt | Opt | Opt | Opt | Opt |
| Kokum Butter | 10-80 | 10-80 | 20-50 | 30-45 | 35-42 |
| Carrier Oil (Avocado or Apricot Kernel) | 5-70 | 5-70 | 15-50 | 25-40 | 28-35 |
| Beeswax | 2-40 | 2-40 | 8-25 | 12-20 | 14-18 |
| Vitamin E (MT-50) | 0.5-10 | 0.1-10 | 0.5-5 | 1-2 | 1.1-1.5 |
| Witch Hazel (Alcohol Free) | Opt | <20 | <5 | <1 | 0.0 |
| Mineral Salts | Opt | <5 | <1 | <0.5 | 0.0 |
| Isopropyl Alcohol | Opt | <10 | <2 | <1 | 0.0 |
| Guar Gum | Opt | <1 | <0.5 | <0.1 | 0.0 |
| Water | Opt | <10 | <2 | <1 | 0.0 |
| Methyl Salicylate | <10 | Opt | <5 | <1 | 0.0 |
| Camphor | <3 | Opt | <2 | <0.5 | 0.0 |
| Parabens | <1 | Opt | <0.5 | <0.1 | 0.0 |
| Gluten | <1 | Opt | <0.5 | <0.1 | 0.0 |
| Total Artificial Colorants | Opt | <1 | <0.5 | <0.1 | 0.0 |
| Total Petroleum Derived Compounds | Opt | <1 | <0.5 | <0.1 | 0.0 |
| Artificial Fragrance | Opt | <1 | <0.5 | <0.1 | 0.0 |
| Carbomers | Opt | <1 | <0.5 | <0.1 | 0.0 |
| Total Alcohols | Opt | <1 | <0.5 | <0.1 | 0.0 |
| FD&C blue #1 | Opt | <1 | <0.5 | <0.1 | 0.0 |
| FD&C yellow #5 | Opt | <1 | <0.5 | <0.1 | 0.0 |
| Petrolatum | Opt | <1 | <0.5 | <0.1 | 0.0 |
| Stearyl Alcohol | Opt | <1 | <0.5 | <0.1 | 0.0 |
| Cetostearyl Alcohol | Opt | <1 | <0.5 | <0.1 | 0.0 |
| SD Alcohol 40 | Opt | <1 | <0.5 | <0.1 | 0.0 |
| Propylene Glycol | Opt | <1 | <0.5 | <0.1 | 0.0 |
| Arnica Montana Flower Extract (Arnica) | Opt | <1 | <0.5 | <0.1 | 0.0 |
| Aloe Barbadensis Leaf Extract (Aloe) | Opt | <1 | <0.5 | <0.1 | 0.0 |
| Boswellia Carterii Resin Extract (Boswellia) | Opt | <1 | <0.5 | <0.1 | 0.0 |
| Calendula Officinalis Flower Extract | Opt | <1 | <0.5 | <0.1 | 0.0 |
| (Calendula) | | | | | |
| Camellia Sinensis Leaf Extract (Green Tea) | Opt | <1 | <0.5 | <0.1 | 0.0 |
| Arctium Lappa Root Extract (Burdock) | Opt | <1 | <0.5 | <0.1 | 0.0 |
| Herbal Extract (ILEX Paraguariensis) | Opt | <1 | <0.5 | <0.1 | 0.0 |
| Carthamus Tinctorius Seed Oil (Safflower) | Opt | <1 | <0.5 | <0.1 | 0.0 |
| Chamomila Recutita Flower Oil (Matricaria) | Opt | <1 | <0.5 | <0.1 | 0.0 |
| Lavandula Angustifolia Oil (Lavender) | Opt | <1 | <0.5 | <0.1 | 0.0 |
| Rosmarinus Officinalis Leaf Oil (Rosemary) | Opt | <1 | <0.5 | <0.1 | 0.0 |
| Allantoin (Comfrey Root) | Opt | <1 | <0.5 | <0.1 | 0.0 |
| Aloe Vera (Aloe Barbadensis) Leaf Juice | Opt | <1 | <0.5 | <0.1 | 0.0 |
| Thyme (Thymus Vulgaris) Oil | Opt | <1 | <0.5 | <0.1 | 0.0 |
| Juniper Berry | Opt | <1 | <0.5 | <0.1 | 0.0 |
| White Tea | Opt | <1 | <0.5 | <0.1 | 0.0 |
| Cedar Leaf Oil | Opt | <1 | <0.5 | <0.1 | 0.0 |
| Nutmeg Oil | Opt | <1 | <0.5 | <0.1 | 0.0 |
| Sorbitan Stearate | Opt | <1 | <0.5 | <0.1 | 0.0 |
| Disteareth-75 IPDI | Opt | <1 | <0.5 | <0.1 | 0.0 |
| Glyceryl Stearate | Opt | <1 | <0.5 | <0.1 | 0.0 |
| Potassium Stearate | Opt | <1 | <0.5 | <0.1 | 0.0 |
| Triethanolamine Stearate | Opt | <1 | <0.5 | <0.1 | 0.0 |
| Stearic Acid | Opt | <1 | <0.5 | <0.1 | 0.0 |
| PET-150 Distearate | Opt | <1 | <0.5 | <0.1 | 0.0 |
| Steareth | Opt | <1 | <0.5 | <0.1 | 0.0 |
| PEG 40 Hydrogentated Caster Oil | Opt | <1 | <0.5 | <0.1 | 0.0 |
| Light Mineral Oil | Opt | <1 | <0.5 | <0.1 | 0.0 |
| Turpentine Oil | Opt | <1 | <0.5 | <0.1 | 0.0 |
| Polysorbate 60 | Opt | <1 | <0.5 | <0.1 | 0.0 |
| Polysorbate 80 | Opt | <1 | <0.5 | <0.1 | 0.0 |
| Glycerin | Opt | <1 | <0.5 | <0.1 | 0.0 |
| Isononanoate PEG-7 caprylic/capric | Opt | <1 | <0.5 | <0.1 | 0.0 |
| Glycerides | | | | | |
| Thymol | Opt | <1 | <0.5 | <0.1 | 0.0 |
| Diisopropyl Adipate | Opt | <1 | <0.5 | <0.1 | 0.0 |
| Aminomethyl Propanol | Opt | <1 | <0.5 | <0.1 | 0.0 |
| DMDM Hydantoin | Opt | <1 | <0.5 | <0.1 | 0.0 |
| Sodium Hydroxide | Opt | <1 | <0.5 | <0.1 | 0.0 |
| Echinacea | Opt | <1 | <0.5 | <0.1 | 0.0 |
| Isopropyl Myristate | Opt | <1 | <0.5 | <0.1 | 0.0 |
| Acrylates/C10 30 Alkyl Acrylate | Opt | <1 | <0.5 | <0.1 | 0.0 |
| Crosspolymer | | | | | |
| Dimethyl Sulfone | Opt | <1 | <0.5 | <0.1 | 0.0 |
| Silicon Dioxide | Opt | <1 | <0.5 | <0.1 | 0.0 |
| Triethanolamine | Opt | <1 | <0.5 | <0.1 | 0.0 |
| Trolamine | Opt | <1 | <0.5 | <0.1 | 0.0 |
| Isoceteh 20 | Opt | <1 | <0.5 | <0.1 | 0.0 |
| Poloxamer 407 | Opt | <1 | <0.5 | <0.1 | 0.0 |
| Quaternium 15 | Opt | <1 | <0.5 | <0.1 | 0.0 |
| Sodium Lauryl Sulfate | Opt | <1 | <0.5 | <0.1 | 0.0 |
| Titanium Dioxide | Opt | <1 | <0.5 | <0.1 | 0.0 |
| Lanolin | Opt | <1 | <0.5 | <0.1 | 0.0 |
| Quaternium 15 | Opt | <1 | <0.5 | <0.1 | 0.0 |
| Sodium Cetearyl Sulfate | Opt | <1 | <0.5 | <0.1 | 0.0 |
| Edetate Disodium | Opt | <1 | <0.5 | <0.1 | 0.0 |
| Ceresin | Opt | <1 | <0.5 | <0.1 | 0.0 |
| Cyclomethicone | Opt | <1 | <0.5 | <0.1 | 0.0 |
| Paraffin | Opt | <1 | <0.5 | <0.1 | 0.0 |
| Microcrystalline Wax | Opt | <1 | <0.5 | <0.1 | 0.0 |

As demonstrated above in TABLE 2, the topical analgesics can comprise various specific ingredients when in the form of a solid stick. For example, in such embodiments, the topical analgesic can comprise beeswax, kokum butter, and/or a carrier oil. In certain embodiments, the topical analgesic in solid stick form can comprise beeswax and/or kokum butter. In other embodiments, the topical analgesic in solid stick form can comprise beeswax and/or a carrier oil. In yet other embodiments, the topical analgesic in solid stick form can comprise kokum butter and/or a carrier oil.

In various embodiments, the topical analgesic in solid stick form can comprise beeswax in an amount in the range of 2 to 40, 8 to 25, or 12 to 20 weight percent.

In various embodiments, the topical analgesic in solid stick form can comprise kokum butter in an amount in the range of 10 to 80, 20 to 50, or 30 to 45 weight percent.

In various embodiments, the topical analgesic in solid stick form can comprise a carrier oil in an amount in the range of 5 to 70, 15 to 50, or 20 to 40 weight percent.

In various embodiments, the topical analgesic in solid stick form can comprise less than 1 weight percent of isopropyl alcohol. In certain embodiments, the topical analgesic in solid stick form can comprise no isopropyl alcohol.

In various embodiments, the topical analgesic in solid stick form can comprise less than 10, 2, or 1 weight percent of an alcohol. In certain embodiments, the topical analgesic in solid stick form can comprise no alcohol.

The topical analgesics of present invention can be used to produce various specific types of solid sticks that are designed to alleviate certain physical ailments. The ranges listed in TABLE 3 are directed to specialized solid sticks formed from the topical analgesic of the present invention.

TABLE 3
Base Stick Formulations

| | Topical Analgesic Formulations | | | |
|---|---|---|---|---|
| INGREDIENTS | 3A | 3B | 3C | 3D |
| Menthol | 8-40 | 10-25 | 12-14 | 12.6 |
| Total Essential Oils (all types) | 0.05-5 | 0.1-4 | 0.75-2 | 1.94 |
| Eucalyptus Oil | 0.01-5 | 0.02-1 | 0.05-0.5 | 0.10 |
| Spearmint Oil | 0.01-5 | 0.1-1.5 | 0.3-1 | 0.64 |
| Lavender Oil | 0.01-5 | 0.05-4 | 0.1-0.5 | 0.26 |
| Lemon Oil | Opt | <1 | <0.5 | 0.0 |
| Peppermint Oil | Opt | <1 | <0.5 | 0.0 |
| Basil Oil | Opt | <1 | <0.5 | 0.0 |
| Wintergreen | Opt | <1 | <0.5 | 0.0 |
| Capsaicin | Opt | <1 | <0.5 | 0.0 |
| Kokum Butter | 10-80 | 20-50 | 30-45 | 37.8 |
| Carrier Oil (Avocado or Apricot Kernel) | 5-70 | 15-50 | 25-40 | 31.5 |
| Beeswax | 2-40 | 2-40 | 12-20 | 15.8 |
| Vitamin E (MT-50) | 0.5-10 | 0.1-10 | 1-2 | 1.3 |
| Witch Hazel (Alcohol Free) | Opt | <20 | <1 | 0.0 |
| Mineral Salts | Opt | <5 | <0.5 | 0.0 |
| Isopropyl Alcohol | Opt | <10 | <1 | 0.0 |
| Guar Gum | Opt | <1 | <0.1 | 0.0 |
| Water | Opt | <10 | <1 | 0.0 |
| Methyl Salicylate | <10 | Opt | <1 | 0.0 |
| Camphor | <3 | Opt | <0.5 | 0.0 |
| Parabens | <1 | Opt | <0.1 | 0.0 |
| Gluten | <1 | Opt | <0.1 | 0.0 |
| Total Artificial Colorants | <3 | <1 | <0.1 | 0.0 |
| Total Petroleum Derived Compounds | Opt | <1 | <0.1 | 0.0 |
| Artificial Fragrance | Opt | <1 | <0.1 | 0.0 |
| Carbomers | <10 | <1 | <0.1 | 0.0 |
| Total Alcohols | Opt | <1 | <0.1 | 0.0 |
| FD&C blue #1 | <1 | <1 | <0.1 | 0.0 |
| FD&C yellow #5 | <1 | <1 | <0.1 | 0.0 |
| Petrolatum | Opt | <1 | <0.1 | 0.0 |
| Stearyl Alcohol | Opt | <1 | <0.1 | 0.0 |
| Cetostearyl Alcohol | Opt | <1 | <0.1 | 0.0 |
| SD Alcohol 40 | Opt | <1 | <0.1 | 0.0 |
| Propylene Glycol | Opt | <1 | <0.1 | 0.0 |
| Arnica Montana Flower Extract (Arnica) | Opt | <1 | <0.1 | 0.0 |
| Aloe Barbadensis Leaf Extract (Aloe) | Opt | <1 | <0.1 | 0.0 |
| Boswellia Carterii Resin Extract (Boswellia) | Opt | <1 | <0.1 | 0.0 |
| Calendula Officinalis Flower Extract | Opt | <1 | <0.1 | 0.0 |
| (Calendula) | | | | |
| Camellia Sinensis Leaf Extract (Green Tea) | Opt | <1 | <0.1 | 0.0 |
| Arctium Lappa Root Extract (Burdock) | Opt | <1 | <0.1 | 0.0 |
| Herbal Extract (ILEX Paraguariensis) | Opt | <1 | <0.1 | 0.0 |
| Carthamus Tinctorius Seed Oil (Safflower) | Opt | <1 | <0.1 | 0.0 |
| Chamomila Recutita Flower Oil (Matricaria) | Opt | <1 | <0.1 | 0.0 |
| Lavandula Angustifolia Oil (Lavender) | Opt | <1 | <0.1 | 0.0 |
| Rosmarinus Officinalis Leaf Oil (Rosemary) | Opt | <1 | <0.1 | 0.0 |
| Allantoin (Comfrey Root) | Opt | <1 | <0.1 | 0.0 |
| Aloe Vera (Aloe Barbadensis) Leaf Juice | Opt | <1 | <0.1 | 0.0 |
| Thyme (Thymus Vulgaris) Oil | Opt | <1 | <0.1 | 0.0 |
| Juniper Berry | Opt | <1 | <0.1 | 0.0 |
| White Tea | Opt | <1 | <0.1 | 0.0 |
| Cedar Leaf Oil | Opt | <1 | <0.1 | 0.0 |
| Nutmeg Oil | Opt | <1 | <0.1 | 0.0 |
| Sorbitan Stearate | Opt | <1 | <0.1 | 0.0 |
| Disteareth-75 IPDI | Opt | <1 | <0.1 | 0.0 |
| Glyceryl Stearate | Opt | <1 | <0.1 | 0.0 |
| Potassium Stearate | Opt | <1 | <0.1 | 0.0 |
| Triethanolamine Stearate | Opt | <1 | <0.1 | 0.0 |
| Stearic Acid | Opt | <1 | <0.1 | 0.0 |
| PET-150 Distearate | Opt | <1 | <0.1 | 0.0 |
| Steareth | Opt | <1 | <0.1 | 0.0 |
| PEG 40 Hydrogentated Caster Oil | Opt | <1 | <0.1 | 0.0 |
| Light Mineral Oil | Opt | <1 | <0.1 | 0.0 |
| Turpentine Oil | Opt | <1 | <0.1 | 0.0 |
| Polysorbate 60 | Opt | <1 | <0.1 | 0.0 |
| Polysorbate 80 | Opt | <1 | <0.1 | 0.0 |
| Glycerin | Opt | <1 | <0.1 | 0.0 |
| Isononanoate PEG-7 caprylic/capric | Opt | <1 | <0.1 | 0.0 |
| Glycerides | | | | |
| Thymol | Opt | <1 | <0.1 | 0.0 |
| Diisopropyl Adipate | Opt | <1 | <0.1 | 0.0 |
| Aminomethyl Propanol | Opt | <1 | <0.1 | 0.0 |
| DMDM Hydantoin | Opt | <1 | <0.1 | 0.0 |
| Sodium Hydroxide | Opt | <1 | <0.1 | 0.0 |
| Echinacea | Opt | <1 | <0.1 | 0.0 |
| Isopropyl Myristate | Opt | <1 | <0.1 | 0.0 |
| Acrylates/C10 30 Alkyl Acrylate | Opt | <1 | <0.1 | 0.0 |
| Crosspolymer | | | | |
| Dimethyl Sulfone | Opt | <1 | <0.1 | 0.0 |
| Silicon Dioxide | Opt | <1 | <0.1 | 0.0 |
| Triethanolamine | Opt | <1 | <0.1 | 0.0 |
| Trolamine | Opt | <1 | <0.1 | 0.0 |
| Isoceteh 20 | Opt | <1 | <0.1 | 0.0 |
| Poloxamer 407 | Opt | <1 | <0.1 | 0.0 |
| Quaternium 15 | Opt | <1 | <0.1 | 0.0 |
| Sodium Lauryl Sulfate | Opt | <1 | <0.1 | 0.0 |
| Titanium Dioxide | Opt | <1 | <0.1 | 0.0 |
| Lanolin | Opt | <1 | <0.1 | 0.0 |
| Quaternium 15 | Opt | <1 | <0.1 | 0.0 |
| Sodium Cetearyl Sulfate | Opt | <1 | <0.1 | 0.0 |
| Edetate Disodium | Opt | <1 | <0.1 | 0.0 |
| Ceresin | Opt | <1 | <0.1 | 0.0 |
| Cyclomethicone | Opt | <1 | <0.1 | 0.0 |
| Paraffin | Opt | <1 | <0.1 | 0.0 |
| Microcrystalline Wax | Opt | <1 | <0.1 | 0.0 |

In various embodiments, the topical analgesic in solid stick form can comprise 0.1 to 4.0 or 0.75 to 2.0 weight percent of one or more essential oils selected from the group consisting of spearmint essential oil, lavender essential oil, and eucalyptus essential oil. In certain embodiments, the topical analgesic in solid stick form can comprise at least two of the following essential oils: spearmint essential oil, lavender essential oil, and eucalyptus essential oil.

In various embodiments, the topical analgesic in solid stick form can comprise spearmint essential oil, lavender essential oil, and eucalyptus essential oil. In certain embodiments, the topical analgesic in solid stick form can comprise spearmint essential oil in an amount in the range of 0.1 to 2 weight percent, lavender essential oil in an amount in the range of 0.1 to 2 weight percent, and/or eucalyptus essential oil in an amount in the range of 0.05 to 1.5 weight percent.

The topical analgesics of present invention can be used to produce capsaicin-containing solid sticks that are designed to alleviate certain physical ailments. The ranges listed in TABLE 4 are directed to specialized solid sticks formed from the topical analgesic of the present invention.

TABLE 4
Red Stick Formulations

| | Topical Analgesic Formulations | | | |
|---|---|---|---|---|
| INGREDIENTS | 4A | 4B | 4C | 4D |
| Menthol | 8-40 | 10-25 | 12-14 | 12.6 |
| Total Essential Oils (all types) | 0.05-5 | 0.1-4 | 0.5-3 | 1.23 |
| Eucalyptus Oil | 0.01-5 | 0.02-1 | 0.04-0.4 | 0.06 |
| Spearmint Oil | 0.01-5 | 0.1-1.5 | 0.2-1 | 0.63 |
| Lavender Oil | 0.01-5 | 0.05-4 | 0.1-0.5 | 0.25 |
| Lemon Oil | 0.01-5 | 0.02-1 | 0.05-0.5 | 0.13 |
| Peppermint Oil | 0.01-5 | 0.05-1 | 0.08-0.5 | 0.19 |
| Basil Oil | Opt | <1 | <0.5 | 0.0 |
| Wintergreen | Opt | <1 | <0.5 | 0.0 |
| Capsaicin | 0.01-5 | 0.05-1 | 0.1-0.5 | 0.19 |
| Kokum Butter | 10-80 | 20-50 | 30-45 | 37.7 |
| Carrier Oil (Avocado or Apricot Kernel) | 5-70 | 15-50 | 25-40 | 31.4 |
| Beeswax | 2-40 | 2-40 | 12-20 | 15.7 |
| Vitamin E (MT-50) | 0.5-10 | 0.1-10 | 1-2 | 1.3 |
| Witch Hazel (Alcohol Free) | Opt | <20 | <1 | 0.0 |
| Mineral Salts | Opt | <5 | <0.5 | 0.0 |
| Isopropyl Alcohol | Opt | <10 | <1 | 0.0 |
| Guar Gum | Opt | <1 | <0.1 | 0.0 |
| Water | Opt | <10 | <1 | 0.0 |
| Methyl Salicylate | <10 | Opt | <1 | 0.0 |
| Camphor | <3 | Opt | <0.5 | 0.0 |
| Parabens | <1 | Opt | <0.1 | 0.0 |
| Gluten | <1 | Opt | <0.1 | 0.0 |
| Total Artificial Colorants | <3 | <1 | <0.1 | 0.0 |
| Total Petroleum Derived Compounds | Opt | <1 | <0.1 | 0.0 |
| Artificial Fragrance | Opt | <1 | <0.1 | 0.0 |
| Carbomers | <10 | <1 | <0.1 | 0.0 |
| Total Alcohols | Opt | <1 | <0.1 | 0.0 |
| FD&C blue #1 | <1 | <1 | <0.1 | 0.0 |
| FD&C yellow #5 | <1 | <1 | <0.1 | 0.0 |
| Petrolatum | Opt | <1 | <0.1 | 0.0 |
| Stearyl Alcohol | Opt | <1 | <0.1 | 0.0 |
| Cetostearyl Alcohol | Opt | <1 | <0.1 | 0.0 |
| SD Alcohol 40 | Opt | <1 | <0.1 | 0.0 |
| Propylene Glycol | Opt | <1 | <0.1 | 0.0 |
| Arnica Montana Flower Extract (Arnica) | Opt | <1 | <0.1 | 0.0 |
| Aloe Barbadensis Leaf Extract (Aloe) | Opt | <1 | <0.1 | 0.0 |
| Boswellia Carterii Resin Extract (Boswellia) | Opt | <1 | <0.1 | 0.0 |
| Calendula Officinalis Flower Extract | Opt | <1 | <0.1 | 0.0 |
| (Calendula) | | | | |
| Camellia Sinensis Leaf Extract (Green Tea) | Opt | <1 | <0.1 | 0.0 |
| Arctium Lappa Root Extract (Burdock) | Opt | <1 | <0.1 | 0.0 |
| Herbal Extract (ILEX Paraguariensis) | Opt | <1 | <0.1 | 0.0 |
| Carthamus Tinctorius Seed Oil (Safflower) | Opt | <1 | <0.1 | 0.0 |
| Chamomila Recutita Flower Oil (Matricaria) | Opt | <1 | <0.1 | 0.0 |
| Lavandula Angustifolia Oil (Lavender) | Opt | <1 | <0.1 | 0.0 |
| Rosmarinus Officinalis Leaf Oil (Rosemary) | Opt | <1 | <0.1 | 0.0 |
| Allantoin (Comfrey Root) | Opt | <1 | <0.1 | 0.0 |
| Aloe Vera (Aloe Barbadensis) Leaf Juice | Opt | <1 | <0.1 | 0.0 |
| Thyme (Thymus Vulgaris) Oil | Opt | <1 | <0.1 | 0.0 |
| Juniper Berry | Opt | <1 | <0.1 | 0.0 |
| White Tea | Opt | <1 | <0.1 | 0.0 |
| Cedar Leaf Oil | Opt | <1 | <0.1 | 0.0 |
| Nutmeg Oil | Opt | <1 | <0.1 | 0.0 |
| Sorbitan Stearate | Opt | <1 | <0.1 | 0.0 |
| Disteareth-75 IPDI | Opt | <1 | <0.1 | 0.0 |
| Glyceryl Stearate | Opt | <1 | <0.1 | 0.0 |
| Potassium Stearate | Opt | <1 | <0.1 | 0.0 |
| Triethanolamine Stearate | Opt | <1 | <0.1 | 0.0 |
| Stearic Acid | Opt | <1 | <0.1 | 0.0 |
| PET-150 Distearate | Opt | <1 | <0.1 | 0.0 |
| Steareth | Opt | <1 | <0.1 | 0.0 |
| PEG 40 Hydrogentated Caster Oil | Opt | <1 | <0.1 | 0.0 |
| Light Mineral Oil | Opt | <1 | <0.1 | 0.0 |
| Turpentine Oil | Opt | <1 | <0.1 | 0.0 |
| Polysorbate 60 | Opt | <1 | <0.1 | 0.0 |
| Polysorbate 80 | Opt | <1 | <0.1 | 0.0 |
| Glycerin | Opt | <1 | <0.1 | 0.0 |
| Isononanoate PEG-7 caprylic/capric | Opt | <1 | <0.1 | 0.0 |
| Glycerides | | | | |
| Thymol | Opt | <1 | <0.1 | 0.0 |
| Diisopropyl Adipate | Opt | <1 | <0.1 | 0.0 |
| Aminomethyl Propanol | Opt | <1 | <0.1 | 0.0 |
| DMDM Hydantoin | Opt | <1 | <0.1 | 0.0 |
| sodium Hydroxide | Opt | <1 | <0.1 | 0.0 |
| Echinace | Opt | <1 | <0.1 | 0.0 |
| Isopropyl Myristate | Opt | <1 | <0.1 | 0.0 |
| Acrylates/C10 30 Alkyl Acrylate | Opt | <1 | <0.1 | 0.0 |
| Crosspolymer | | | | |
| Dimethyl Sulfone | Opt | <1 | <0.1 | 0.0 |
| Silicon Dioxide | Opt | <1 | <0.1 | 0.0 |
| Triethanolamine | Opt | <1 | <0.1 | 0.0 |
| Trolamine | Opt | <1 | <0.1 | 0.0 |
| Isoceteh 20 | Opt | <1 | <0.1 | 0.0 |
| Poloxamer 407 | Opt | <1 | <0.1 | 0.0 |
| Quaternium 15 | Opt | <1 | <0.1 | 0.0 |
| Sodium Lauryl Sulfate | Opt | <1 | <0.1 | 0.0 |
| Titanium Dioxide | Opt | <1 | <0.1 | 0.0 |
| Lanolin | Opt | <1 | <0.1 | 0.0 |
| Quaternium 15 | Opt | <1 | <0.1 | 0.0 |
| Sodium Cetearyl Sulfate | Opt | <1 | <0.1 | 0.0 |
| Edetate Disodium | Opt | <1 | <0.1 | 0.0 |
| Ceresin | Opt | <1 | <0.1 | 0.0 |
| Cyclomethicone | Opt | <1 | <0.1 | 0.0 |
| Paraffin | Opt | <1 | <0.1 | 0.0 |
| Microcrystalline Wax | Opt | <1 | <0.1 | 0.0 |

In various embodiments, the topical analgesic in solid stick form can comprise capsaicin in an amount in the range of 0.01 to 5, 0.05 to 1, or 0.1 to 0.5 weight percent.

The topical analgesics of present invention can be used to produce essential oil-containing solid sticks that are designed to alleviate certain physical ailments. The ranges listed in TABLE 5 are directed to specialized solid sticks formed from the topical analgesic of the present invention.

TABLE 5
Green Stick Formulations

| | Topical Analgesic Formulations | | | |
|---|---|---|---|---|
| INGREDIENTS | 5A | 5B | 5C | 5D |
| Menthol | 8-40 | 10-25 | 12-14 | 12.6 |
| Total Essential Oils (all types) | 0.05-5 | 0.1-4 | 0.75-2 | 1.1 |
| Eucalyptus Oil | Opt | <1 | <0.5 | 0.0 |
| Spearmint Oil | Opt | <1 | <0.5 | 0.0 |
| Lavender Oil | Opt | <1 | <0.5 | 0.0 |
| Lemon Oil | 0.01-5 | 0.05-1 | 0.1-0.5 | 0.2 |
| Peppermint Oil | 0.01-5 | 0.05-1 | 0.2-0.8 | 0.4 |
| Basil Oil | 0.01-5 | 0.05-1 | 0.1-0.5 | 0.2 |
| Wintergreen | 0.01-5 | 0.05-1 | 0.15-0.6 | 0.3 |
| Capsaicin | Opt | <1 | <0.5 | 0.0 |
| Kokum Butter | 10-80 | 20-50 | 30-45 | 37.8 |
| Carrier Oil (Avocado or Apricot Kernel) | 5-70 | 15-50 | 25-40 | 31.5 |
| Beeswax | 2-40 | 2-40 | 12-20 | 15.7 |
| Vitamin E (MT-50) | 0.5-10 | 0.1-10 | 1-2 | 1.3 |
| Witch Hazel (Alcohol Free) | Opt | <20 | <1 | 0.0 |
| Mineral Salts | Opt | <5 | <0.5 | 0.0 |
| Isopropyl Alcohol | Opt | <10 | <1 | 0.0 |
| Guar Gum | Opt | <1 | <0.1 | 0.0 |
| Water | Opt | <10 | <1 | 0.0 |
| Methyl Salicylate | <10 | Opt | <1 | 0.0 |
| Camphor | <3 | Opt | <0.5 | 0.0 |
| Parabens | <1 | Opt | <0.1 | 0.0 |
| Gluten | <1 | Opt | <0.1 | 0.0 |
| Total Artificial Colorants | <3 | <1 | <0.1 | 0.0 |
| Total Petroleum Derived Compounds | Opt | <1 | <0.1 | 0.0 |
| Artificial Fragrance | Opt | <1 | <0.1 | 0.0 |
| Carbomers | <10 | <1 | <0.1 | 0.0 |
| Total Alcohols | Opt | <1 | <0.1 | 0.0 |
| FD&C blue #1 | <1 | <1 | <0.1 | 0.0 |
| FD&C yellow #5 | <1 | <1 | <0.1 | 0.0 |
| Petrolatum | Opt | <1 | <0.1 | 0.0 |
| Stearyl Alcohol | Opt | <1 | <0.1 | 0.0 |
| Cetostearyl Alcohol | Opt | <1 | <0.1 | 0.0 |
| SD Alcohol 40 | Opt | <1 | <0.1 | 0.0 |
| Propylene Glycol | Opt | <1 | <0.1 | 0.0 |
| Arnica Montana Flower Extract (Arnica) | Opt | <1 | <0.1 | 0.0 |
| Aloe Barbadensis Leaf Extract (Aloe) | Opt | <1 | <0.1 | 0.0 |
| Boswellia Carterii Resin Extract (Boswellia) | Opt | <1 | <0.1 | 0.0 |
| Calendula Officinalis Flower Extract | Opt | <1 | <0.1 | 0.0 |
| (Calendula) | | | | |
| Camellia Sinensis Leaf Extract (Green Tea) | Opt | <1 | <0.1 | 0.0 |
| Arctium Lappa Root Extract (Burdock) | Opt | <1 | <0.1 | 0.0 |
| Herbal Extract (ILEX Paraguariensis) | Opt | <1 | <0.1 | 0.0 |
| Carthamus Tinctorius Seed Oil (Safflower) | Opt | <1 | <0.1 | 0.0 |
| Chamomila Recutita Flower Oil (Matricaria) | Opt | <1 | <0.1 | 0.0 |
| Lavandula Angustifolia Oil (Lavender) | Opt | <1 | <0.1 | 0.0 |
| Rosmarinus Officinalis Leaf Oil (Rosemary) | Opt | <1 | <0.1 | 0.0 |
| Allantoin (Comfrey Root) | Opt | <1 | <0.1 | 0.0 |
| Aloe Vera (Aloe Barbadensis) Leaf Juice | Opt | <1 | <0.1 | 0.0 |
| Thyme (Thymus Vulgaris) Oil | Opt | <1 | <0.1 | 0.0 |
| Juniper Berry | Opt | <1 | <0.1 | 0.0 |
| White Tea | Opt | <1 | <0.1 | 0.0 |
| Cedar Leaf Oil | Opt | <1 | <0.1 | 0.0 |
| Nutmeg Oil | Opt | <1 | <0.1 | 0.0 |
| Sorbitan Stearate | Opt | <1 | <0.1 | 0.0 |
| Disteareth-75 IPDI | Opt | <1 | <0.1 | 0.0 |
| Glyceryl Stearate | Opt | <1 | <0.1 | 0.0 |
| Potassium Stearate | Opt | <1 | <0.1 | 0.0 |
| Triethanolamine Stearate | Opt | <1 | <0.1 | 0.0 |
| Stearic Acid | Opt | <1 | <0.1 | 0.0 |
| PET-150 Distearate | Opt | <1 | <0.1 | 0.0 |
| Steareth | Opt | <1 | <0.1 | 0.0 |
| PEG 40 Hydrogentated Caster Oil | Opt | <1 | <0.1 | 0.0 |
| Light Mineral Oil | Opt | <1 | <0.1 | 0.0 |
| Turpentine Oil | Opt | <1 | <0.1 | 0.0 |
| Polysorbate 60 | Opt | <1 | <0.1 | 0.0 |
| Polysorbate 80 | Opt | <1 | <0.1 | 0.0 |
| Glycerin | Opt | <1 | <0.1 | 0.0 |
| Isononanoate PEG-7 caprylic/capric | Opt | <1 | <0.1 | 0.0 |
| Glycerides | | | | |
| Thymol | Opt | <1 | <0.1 | 0.0 |
| Diisopropyl Adipate | Opt | <1 | <0.1 | 0.0 |
| Aminomethyl Propanol | Opt | <1 | <0.1 | 0.0 |
| DMDM Hydantoin | Opt | <1 | <0.1 | 0.0 |
| sodium Hydroxide | Opt | <1 | <0.1 | 0.0 |
| Echinace | Opt | <1 | <0.1 | 0.0 |
| Isopropyl Myristate | Opt | <1 | <0.1 | 0.0 |
| Acrylates/C10 30 Alkyl Acrylate | Opt | <1 | <0.1 | 0.0 |
| Crosspolymer | | | | |
| Dimethyl Sulfone | Opt | <1 | <0.1 | 0.0 |
| Silicon Dioxide | Opt | <1 | <0.1 | 0.0 |
| Triethanolamine | Opt | <1 | <0.1 | 0.0 |
| Trolamine | Opt | <1 | <0.1 | 0.0 |
| Isoceteh 20 | Opt | <1 | <0.1 | 0.0 |
| Poloxamer 407 | Opt | <1 | <0.1 | 0.0 |
| Quaternium 15 | Opt | <1 | <0.1 | 0.0 |
| Sodium Lauryl Sulfate | Opt | <1 | <0.1 | 0.0 |
| Titanium Dioxide | Opt | <1 | <0.1 | 0.0 |
| Lanolin | Opt | <1 | <0.1 | 0.0 |
| Quaternium 15 | Opt | <1 | <0.1 | 0.0 |
| Sodium Cetearyl Sulfate | Opt | <1 | <0.1 | 0.0 |
| Edetate Disodium | Opt | <1 | <0.1 | 0.0 |
| Ceresin | Opt | <1 | <0.1 | 0.0 |
| Cyclomethicone | Opt | <1 | <0.1 | 0.0 |
| Paraffin | Opt | <1 | <0.1 | 0.0 |
| Microcrystalline Wax | Opt | <1 | <0.1 | 0.0 |

In various embodiments, the topical analgesic in solid stick form can comprise 0.1 to 4.0 or 0.75 to 2.0 weight percent of one or more essential oils selected from the group consisting of lemon essential oil, peppermint essential oil, basil essential oil, and wintergreen essential oil. In certain embodiments, the topical analgesic in solid stick form can comprise at least two or three of the following essential oils: lemon essential oil, peppermint essential oil, basil essential oil, and wintergreen essential oil. In one or more embodiments, the topical analgesic in solid stick form can comprise lemon essential oil, peppermint essential oil, basil essential oil, and wintergreen essential oil. In other embodiments, the topical analgesic in solid stick form can comprise lemon essential oil in an amount in the range of 0.05 to 1 weight percent, peppermint essential oil in an amount in the range of 0.05 to 1 weight percent, basil essential oil in an amount in the range of 0.05 to 1 weight percent, and/or wintergreen essential oil in an amount in the range of 0.05 to 1 weight percent.

The topical analgesics of present invention can be used to produce gels that are designed to alleviate certain physical ailments. The ranges listed in TABLE 6 are directed to specialized gels formed from the topical analgesic of the present invention.

TABLE 6
Gel Formulations

| | Topical Analgesic Formulations | | | |
|---|---|---|---|---|
| INGREDIENTS | 6A | 6B | 6C | 6D |
| Menthol | 8-40 | 10-25 | 12-14 | 12.4 |
| Total Essential Oils (all types) | 0.05-5 | 0.1-4 | 0.5-3 | 1.73 |
| Eucalyptus Oil | 0.01-5 | 0.02-1 | 0.05-0.5 | 0.21 |
| Spearmint Oil | 0.05-5 | 0.1-2.5 | 0.2-1.5 | 0.85 |
| Lavender Oil | 0.01-5 | 0.05-4 | 0.1-0.5 | 0.25 |
| Lemon Oil | 0.01-5 | 0.05-1 | 0.1-0.5 | 0.21 |
| Peppermint Oil | 0.01-5 | 0.05-1 | 0.1-0.5 | 0.21 |
| Basil Oil | Opt | <1 | <0.5 | 0.0 |
| Wintergreen | Opt | <1 | <0.5 | 0.0 |
| Capsaicin | Opt | <1 | <0.5 | 0.0 |
| Kokum Butter | Opt | <10 | <1 | 0.0 |
| Carrier Oil (Avocado or Apricot Kernel) | Opt | <10 | <1 | 0.0 |
| Beeswax | Opt | <10 | <1 | 0.0 |
| Vitamin E (MT-50) | Opt | <1 | <0.5 | 0.0 |
| Witch Hazel (Alcohol Free) | 20-80 | 35-65 | 45-60 | 52.2 |
| Mineral Salts | 1-25 | 2-15 | 4-10 | 7.5 |
| Isopropyl Alcohol | 2-50 | 5-25 | 8-16 | 12.4 |
| Guar Gum | 0.05-12 | 0.1-8 | 0.5-4 | 1.2 |
| Water | 2-50 | 5-25 | 8-16 | 12.4 |
| Methyl Salicylate | <10 | Opt | <1 | 0.0 |
| Camphor | <3 | Opt | <0.5 | 0.0 |
| Parabens | <1 | Opt | <0.1 | 0.0 |
| Gluten | <1 | Opt | <0.1 | 0.0 |
| Total Artificial Colorants | <3 | <1 | <0.1 | 0.0 |
| Total Petroleum Derived Compounds | Opt | <1 | <0.1 | 0.0 |
| Artificial Fragrance | Opt | <1 | <0.1 | 0.0 |
| Carbomers | <10 | <1 | <0.1 | 0.0 |
| Total Alcohols | Opt | <1 | <0.1 | 0.0 |
| FD&C blue #1 | <1 | <1 | <0.1 | 0.0 |
| FD&C yellow #5 | <1 | <1 | <0.1 | 0.0 |
| Petrolatum | Opt | <1 | <0.1 | 0.0 |
| Stearyl Alcohol | Opt | <1 | <0.1 | 0.0 |
| Cetostearyl Alcohol | Opt | <1 | <0.1 | 0.0 |
| SD Alcohol 40 | Opt | <1 | <0.1 | 0.0 |
| Propylene Glycol | Opt | <1 | <0.1 | 0.0 |
| Arnica Montana Flower Extract (Arnica) | Opt | <1 | <0.1 | 0.0 |
| Aloe Barbadensis Leaf Extract (Aloe) | Opt | <1 | <0.1 | 0.0 |
| Boswellia Carterii Resin Extract (Boswellia) | Opt | <1 | <0.1 | 0.0 |
| Calendula Officinalis Flower Extract | Opt | <1 | <0.1 | 0.0 |
| (Calendula) | | | | |
| Camellia Sinensis Leaf Extract (Green Tea) | Opt | <1 | <0.1 | 0.0 |
| Arctium Lappa Root Extract (Burdock) | Opt | <1 | <0.1 | 0.0 |
| Herbal Extract (ILEX Paraguariensis) | Opt | <1 | <0.1 | 0.0 |
| Carthamus Tinctorius Seed Oil (Safflower) | Opt | <1 | <0.1 | 0.0 |
| Chamomila Recutita Flower Oil (Matricaria) | Opt | <1 | <0.1 | 0.0 |
| Lavandula Angustifolia Oil (Lavender) | Opt | <1 | <0.1 | 0.0 |
| Rosmarinus Officinalis Leaf Oil (Rosemary) | Opt | <1 | <0.1 | 0.0 |
| Allantoin (Comfrey Root) | Opt | <1 | <0.1 | 0.0 |
| Aloe Vera (Aloe Barbadensis) Leaf Juice | Opt | <1 | <0.1 | 0.0 |
| Thyme (Thymus Vulgaris) Oil | Opt | <1 | <0.1 | 0.0 |
| Juniper Berry | Opt | <1 | <0.1 | 0.0 |
| White Tea | Opt | <1 | <0.1 | 0.0 |
| Cedar Leaf Oil | Opt | <1 | <0.1 | 0.0 |
| Nutmeg Oil | Opt | <1 | <0.1 | 0.0 |
| Sorbitan Stearate | Opt | <1 | <0.1 | 0.0 |
| Disteareth-75 IPDI | Opt | <1 | <0.1 | 0.0 |
| Glyceryl Stearate | Opt | <1 | <0.1 | 0.0 |
| Potassium Stearate | Opt | <1 | <0.1 | 0.0 |
| Triethanolamine Stearate | Opt | <1 | <0.1 | 0.0 |
| Stearic Acid | Opt | <1 | <0.1 | 0.0 |
| PET-150 Distearate | Opt | <1 | <0.1 | 0.0 |
| Steareth | Opt | <1 | <0.1 | 0.0 |
| PEG 40 Hydrogentated Caster Oil | Opt | <1 | <0.1 | 0.0 |
| Light Mineral Oil | Opt | <1 | <0.1 | 0.0 |
| Turpentine Oil | Opt | <1 | <0.1 | 0.0 |
| Polysorbate 60 | Opt | <1 | <0.1 | 0.0 |
| Polysorbate 80 | Opt | <1 | <0.1 | 0.0 |
| Glycerin | Opt | <1 | <0.1 | 0.0 |
| Isononanoate PEG-7 caprylic/capric | Opt | <1 | <0.1 | 0.0 |
| Glycerides | | | | |
| Thymol | Opt | <1 | <0.1 | 0.0 |
| Diisopropyl Adipate | Opt | <1 | <0.1 | 0.0 |
| Aminomethyl Propanol | Opt | <1 | <0.1 | 0.0 |
| DMDM Hydantoin | Opt | <1 | <0.1 | 0.0 |
| sodium Hydroxide | Opt | <1 | <0.1 | 0.0 |
| Echinace | Opt | <1 | <0.1 | 0.0 |
| Isopropyl Myristate | Opt | <1 | <0.1 | 0.0 |
| Acrylates/C10 30 Alkyl Acrylate | Opt | <1 | <0.1 | 0.0 |
| Crosspolymer | | | | |
| Dimethyl Sulfone | Opt | <1 | <0.1 | 0.0 |
| Silicon Dioxide | Opt | <1 | <0.1 | 0.0 |
| Triethanolamine | Opt | <1 | <0.1 | 0.0 |
| Trolamine | Opt | <1 | <0.1 | 0.0 |
| Isoceteh 20 | Opt | <1 | <0.1 | 0.0 |
| Poloxamer 407 | Opt | <1 | <0.1 | 0.0 |
| Quaternium 15 | Opt | <1 | <0.1 | 0.0 |
| Sodium Lauryl Sulfate | Opt | <1 | <0.1 | 0.0 |
| Titanium Dioxide | Opt | <1 | <0.1 | 0.0 |
| Lanolin | Opt | <1 | <0.1 | 0.0 |
| Quaternium 15 | Opt | <1 | <0.1 | 0.0 |
| Sodium Cetearyl Sulfate | Opt | <1 | <0.1 | 0.0 |
| Edetate Disodium | Opt | <1 | <0.1 | 0.0 |
| Ceresin | Opt | <1 | <0.1 | 0.0 |
| Cyclomethicone | Opt | <1 | <0.1 | 0.0 |
| Paraffin | Opt | <1 | <0.1 | 0.0 |
| Microcrystalline Wax | Opt | <1 | <0.1 | 0.0 |

In various embodiments, the topical analgesic in gel form can comprise alcohol-free witch hazel in an amount in the range of 20 to 80, 35 to 65, or 45 to 60 weight percent.

In various embodiments, the topical analgesic in gel form can comprise water in an amount in the range of 2 to 50 or 8 to 16 weight percent.

In various embodiments, the topical analgesic in gel form can comprise isopropyl alcohol in an amount in the range of 2 to 50 or 8 to 16 weight percent.

In various embodiments, the topical analgesic in gel form can comprise kokum butter, a carrier oil, and beeswax in an amount of not more than 10, 5, or 1 weight percent.

The topical analgesics described herein can be used to treat various physical ailments afflicting a person. Generally, the topical analgesic of the present invention may be contacted with the skin of a person that is afflicted with a physical ailment, such as dermatitis, muscle pain, joint pain, or arthritis. The topical analgesics described herein may be applied to the skin surface afflicted with the physical ailment and allowed to remain on the skin until symptoms of the ailment are alleviated.

The preferred forms of the invention described above are to be used as illustration only, and should not be used in a limiting sense to interpret the scope of the present invention. Modifications to the exemplary embodiments, set forth above, could be readily made by those skilled in the art without departing from the spirit of the present invention.

The inventors hereby state their intent to rely on the Doctrine of Equivalents to determine and assess the reasonably fair scope of the present invention as it pertains to any apparatus not materially departing from but outside the literal scope of the invention as set forth in the following claims.

### DEFINITIONS

It should be understood that the following is not intended to be an exclusive list of defined terms. Other definitions may be provided in the foregoing description, such as, for example, when accompanying the use of a defined term in context.

As used herein, the terms “a,” “an,” and “the” mean one or more.

As used herein, the term “and/or,” when used in a list of two or more items, means that any one of the listed items can be employed by itself or any combination of two or more of the listed items can be employed. For example, if a composition is described as containing components A, B, and/or C, the composition can contain A alone; B alone; C alone; A and B in combination; A and C in combination, B and C in combination; or A, B, and C in combination.

As used herein, the terms “comprising,” “comprises,” and “comprise” are open-ended transition terms used to transition from a subject recited before the term to one or more elements recited after the term, where the element or elements listed after the transition term are not necessarily the only elements that make up the subject.

As used herein, the terms “having,” “has,” and “have” have the same open-ended meaning as “comprising,” “comprises,” and “comprise” provided above.

As used herein, the terms “including,” “include,” and “included” have the same open-ended meaning as “comprising,” “comprises,” and “comprise” provided above.

### Numerical Ranges

The present description uses numerical ranges to quantify certain parameters relating to the invention. It should be understood that when numerical ranges are provided, such ranges are to be construed as providing literal support for claim limitations that only recite the lower value of the range as well as claim limitations that only recite the upper value of the range. For example, a disclosed numerical range of 10 to 100 provides literal support for a claim reciting “greater than 10” (with no upper bounds) and a claim reciting “less than 100” (with no lower bounds).

What is claimed is:

1. A topical analgesic comprising:
8 to 40 weight percent menthol; and
0.05 to 5 weight percent of one or more essential oils selected from the group consisting of spearmint essential oil, lavender essential oil, eucalyptus essential oil, lemon essential oil, peppermint essential oil, basil essential oil, and wintergreen essential oil,
wherein said topical analgesic comprises less than 1 weight percent parabens and less than 1 weight percent gluten,
wherein said topical analgesic meets one or more of the following criteria—
(i) said topical analgesic comprises less than 10 percent methyl salicylate,
(ii) said topical analgesic comprises less than 3 percent camphor.

2. The topical analgesic of claim 1, wherein said topical analgesic comprises menthol in an amount in the range of from 10 to 25 weight percent.

3. The topical analgesic of claim 1, wherein said topical analgesic comprises at least three of the following essential oils: spearmint essential oil, lavender essential oil, eucalyptus essential oil, lemon essential oil, peppermint essential oil, basil essential oil, and wintergreen essential oil.

4. The topical analgesic of claim 1, wherein said topical analgesic comprises 0.75 to 2.0 weight percent of one or more essential oils selected from the group consisting of spearmint essential oil, lavender essential oil, eucalyptus essential oil, lemon essential oil, peppermint essential oil, basil essential oil, and wintergreen essential oil.

5. The topical analgesic of claim 1, wherein criteria (i) is met.

6. The topical analgesic of claim 5, wherein said topical analgesic comprises methyl salicylate in an amount of less than 1 weight percent.

7. The topical analgesic of claim 1, wherein criteria (ii) is met.

8. The topical analgesic of claim 7, wherein said topical analgesic comprises camphor in an amount of less than 0.5 weight percent.

9. The topical analgesic of claim 1, wherein criteria (i) and criteria (ii) are met.

10. The topical analgesic of claim 9, wherein said topical analgesic comprises no methyl salicylate and no camphor.

11. The topical analgesic of claim 1, wherein said topical analgesic comprises no alcohol.

12. The topical analgesic of claim 1, wherein said topical analgesic comprises parabens in an amount of less than 0.1 weight percent.

13. The topical analgesic of claim 1, wherein said topical analgesic comprises gluten in an amount of less than 0.1 weight percent.

14. The topical analgesic of claim 1, wherein said topical analgesic is in the form of a gel or a solid stick.

15. A solid stick topical analgesic, said solid stick topical analgesic comprising:
(i) 8 to 40 weight percent menthol;
(ii) 0.05 to 5 weight percent of one or more essential oils selected from the group consisting of spearmint essential oil, lavender essential oil, eucalyptus essential oil, lemon essential oil, peppermint essential oil, basil essential oil, and wintergreen essential oil;
(iii) 10 to 80 weight percent of kokum butter;
(iv) 5 to 70 weight percent of a carrier oil;
(v) 2 to 40 weight percent of a beeswax;
(vi) 0.5 to 10 weight percent of Vitamin E;
(vii) less than 10 weight percent of methyl salicylate;
(viii) less than 1 weight percent of parabens;
(ix) less than 1 weight percent of gluten; and
(x) less than 3 percent of camphor.

16. The solid stick topical analgesic of claim 15, wherein said topical analgesic comprises:
(i) 10 to 25 weight percent of menthol;
(ii) 20 to 50 weight percent of kokum butter;
(iii) 12 to 20 weight percent of beeswax; and
(iv) 15 to 50 weight percent of carrier oil.

17. The solid stick topical analgesic of claim 15, wherein said solid stick topical analgesic comprises:
(i) 0.1 to 1.5 weight percent of spearmint essential oil;
(ii) 0.05 to 4 weight percent of lavender essential oil; and
(iii) 0.02 to 1 weight percent of eucalyptus essential oil.

18. A packaged topical analgesic comprising (a) a topical analgesic and (b) a container for housing said topical analgesic, wherein said topical analgesic comprises:
8 to 40 weight percent menthol, and
0.05 to 5 weight percent of one or more essential oils selected from the group consisting of spearmint essential oil, lavender essential oil, eucalyptus essential oil, lemon essential oil, peppermint essential oil, basil essential oil, and wintergreen essential oil,
wherein said topical analgesic comprises less than 1 weight percent parabens and less than 1 weight percent gluten,
wherein said topical analgesic meets one or more of the following criteria—
(i) said topical analgesic comprises less than 10 percent methyl salicylate,
(ii) said topical analgesic comprises less than 3 percent camphor.

19. The packaged topical analgesic of claim 18, wherein said topical analgesic is in the form of a solid stick.

20. The packaged topical analgesic of claim 18, wherein criteria (i) and criteria (ii) are met.
